(19) United States
(12) Patent Application Publication
(10) Pub. No.: US 2024/0394575 A1
(43) Pub. Date: Nov. 28, 2024

(54) INFORMATION PROCESSING APPARATUS, INFERENCE APPARATUS, MACHINE-LEARNING APPARATUS, INFORMATION PROCESSING METHOD, INFERENCE METHOD, AND MACHINE-LEARNING METHOD

(71) Applicant: EBARA CORPORATION, Tokyo (JP)

(72) Inventor: Seiji MURATA, Tokyo (JP)

(73) Assignee: EBARA CORPORATION, Tokyo (JP)

(21) Appl. No.: 18/696,230

(22) PCT Filed: Jul. 11, 2022
(86) PCT No.: PCT/JP2022/027180

§ 371 Date: Mar. 27, 2024

(30) Foreign Application Priority Data
Oct. 4, 2021 (JP) 2021-163358

(51) Int. Cl. G06N 5/046; G06N 3/09

(52) U.S. Cl. CPC G06N 5/046; G01N 33/0095; G06N 3/09

(57) ABSTRACT

An information processing apparatus (5) includes an information acquisition section (500) configured to acquire crack occurrence state information including crack state information indicating crack state of a substrate that has been cracked and device state information indicating a state of a polishing unit when the substrate processing process is performed on the cracked substrate; and a crack occurrence process identifying section (501) configured to identify a process that causes the crack in the substrate by inputting the crack occurrence state information acquired by the information acquisition section (500) to a learning model (11) in response to the occurrence of the crack in the substrate. The learning model (11) has been generated by machine learning that causes the learning model (11) to learn a correlation between the crack occurrence state information and crack occurrence process information indicating the process that causes the crack in the substrate.

### TECHNICAL FIELD

The present invention relates to an information processing apparatus, an inference apparatus, a machine-learning apparatus, an information processing method, an inference method, and a machine-learning method.

### BACKGROUND ART

A substrate processing apparatus that performs chemical mechanical polishing (CMP) is known as a type of substrate processing apparatus that performs various processes on a substrate, such as semiconductor wafer. The substrate that is subjected to various processing in the substrate processing apparatus has a thin plate shape. Therefore, the substrate can be cracked not only during the polishing process of the substrate, but also during the process of transporting the substrate between units. (for example, see Patent Document 1 and Patent Document 2).

### CITATION LIST

### Patent Literature

Patent document 1: Japanese laid-open patent publication No. 2020-188233
Patent document 2: Japanese laid-open patent publication No. 2000-223380

### SUMMARY OF INVENTION

### Technical Problem

If the substrate is cracked for some reason during the substrate processing process performed by the substrate processing apparatus, it is necessary to inspect each unit of the substrate processing apparatus and check various device parameters to which the substrate processing apparatus refers during operation. In particular, the substrate may be cracked in a polishing unit for performing the polishing process or in a substrate transporting unit for transporting the substrate to the polishing unit. If it remains unknown which of units the substrate was cracked, the above-mentioned work cannot be carried out effectively, and there is a possibility that cracking of a substrate will occur again. In addition, analyzing the cause of cracking of the substrate is highly dependent on an experience and knowledge of a user of the substrate processing apparatus. If the analysis is not appropriate, more serious defect may occur or productivity may be lowered.

In view of the above-mentioned drawbacks, it is an object of the present invention to provide an information processing apparatus, an inference apparatus, a machine-learning apparatus, an information processing method, an inference method, and a machine-learning method that analyze a cause of cracking of a substrate in a substrate processing apparatus without depending on a user's experience or knowledge.

### Solution to Problem

In order to achieve the above object, an information processing apparatus comprises: an information acquisition section configured to acquire crack occurrence state information including crack state information and device state information, the crack state information indicating crack state of a substrate that has been cracked in a substrate processing process performed by a substrate processing device including a polishing unit configured to perform a polishing process on the substrate and a substrate transport unit configured to transport the substrate to and from the polishing unit, the device state information indicating a state of the polishing unit when the substrate processing process is performed on the cracked substrate; and a crack occurrence process identifying section configured to identify a process that causes the crack in the substrate by inputting the crack occurrence state information acquired by the information acquisition section to a learning model in response to the occurrence of the crack in the substrate, the learning model having been generated by machine learning that causes the learning model to learn a correlation between the crack occurrence state information and crack occurrence process information indicating the process that causes the crack in the substrate, the process being among processes included in the substrate processing process.

### Advantageous Effects of Invention

According to the information processing apparatus according to the embodiment of the present invention, the cracking-occurrence-state information including the cracking-state information and the device-state information is obtained in response to occurrence of cracking of the substrate. The cracking-occurrence-state information is input to the learning model, so that the cause of the cracking of the substrate is identified. As a result, a user can deal with the cracking of the substrate quickly and appropriately without relying on experience and knowledge of the user.

Objects, configurations, and effects other than those described above will be made clear in detailed descriptions of the invention described below.

### BRIEF DESCRIPTION OF DRAWINGS

FIG. 1 is an overall configuration diagram showing an example of a substrate processing system 1;

FIG. 2 is a plan view showing an example of a substrate processing device 2;

FIG. 3 is a perspective view showing an example of first to fourth polishing sections 22A to 22D;

FIG. 4 is a cross-sectional view schematically showing an example of a top ring 221;

FIG. 5 is a plan view schematically showing an example of first and second linear transporters 230A and 230B;

FIG. 6 is a front view schematically showing an example of first and second linear transporters 230A and 230B;

FIG. 7A is a schematic diagram showing an example of a substrate receiving process;

FIG. 7B is a schematic diagram showing an example of a substrate receiving process;

FIG. 7C is a schematic diagram showing an example of a substrate receiving process;

FIG. 8A is a schematic diagram showing an example of a substrate delivery process;

FIG. 8B is a schematic diagram showing an example of a substrate delivery process;

FIG. 8C is a schematic diagram showing an example of a substrate delivery process.

FIG. 9 is a block diagram showing an example of substrate processing device 2;

FIG. 10 is a timing chart showing an example of a substrate processing process performed by the substrate processing device 2;

FIG. 11 is a hardware configuration diagram showing an example of a computer 900;

FIG. 12 is a data configuration diagram showing an example of history information 30 managed by a database device 3;

FIG. 13 is a block diagram showing an example of a machine-learning device 4;

FIG. 14 is a diagram showing an example of a learning model 10 and learning data 11;

FIG. 15 is a flowchart illustrating an example of a machine-learning method performed by the machine-learning device 4;

FIG. 16 is a block diagram showing an example of an information processing device 5;

FIG. 17 is a functional explanatory diagram showing an example of the information processing device 5; and

FIG. 18 is a flowchart illustrating an example of an information processing method performed by the information processing device 5.

### DESCRIPTION OF EMBODIMENTS

Embodiments for practicing the present invention will be described below with reference to the drawings. In the following descriptions, scope necessary for the descriptions to achieve the object of the present invention will be schematically shown, scope necessary for the descriptions of relevant parts of the present invention will be mainly described, and parts omitted from the descriptions will be based on known technology.

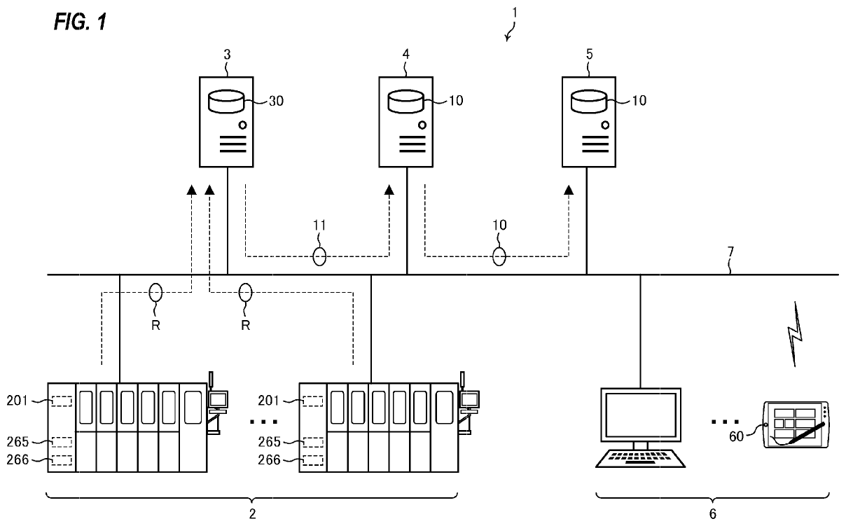

FIG. 1 is an overall configuration diagram showing an example of a substrate processing system 1. As shown in FIG. 1, the substrate processing system 1 according to the present embodiment functions as a system configured to manage a substrate processing in which a chemical mechanical polishing (hereinafter referred to as “polishing process”) is performed on a substrate (hereinafter referred to as “wafer”) W, such as a semiconductor wafer.

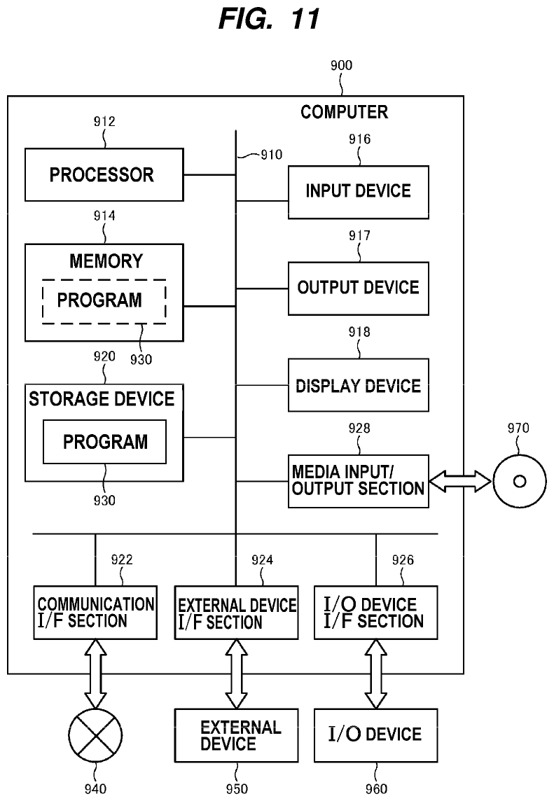

The substrate processing system 1 includes, as its main components, substrate processing devices 2, a database device 3, a machine-learning device 4, an information processing device 5, and a user terminal device 6. Each of the devices 2 to 6 is configured with, for example, a general-purpose or dedicated computer (see FIG. 11 described later). The devices 2 to 6 are coupled to a wired or wireless network 7 so as to be able to mutually transmit and receive various data (some data are shown in FIG. 1 with dotted arrows). It is noted that that the number of devices 2 to 6 and the connection configuration of the network 7 are not limited to the example shown in FIG. 1 and may be changed as appropriate.

Each substrate processing device 2 is an apparatus configured to perform the polishing process on a wafer W to planarize a surface of the wafer W. The substrate processing device 2 is composed of modules and is configured to perform a series of polishing operations on one or more wafers W, such as loading, polishing, cleaning, drying, film-thickness measuring, and unloading. During the operations, the substrate processing device 2 controls operations of the units while referring to device setting information 265 including device parameters that have been set for the modules and substrate recipe information 266 that determines polishing conditions in the polishing process. If the state of each module meets a predetermined alarm-generating condition, the substrate processing device 2 generates an alarm. The substrate processing device 2 further includes a camera 201 disposed at a position where the camera 201 can generate an image of the wafer W.

The substrate processing device 2 is configured to transmit various reports R to the database device 3, the user terminal device 6, etc. according to the operation of each unit. The various reports R include, for example, process information that identifies a wafer W on which each process is performed, device-state information that indicates a state of each unit when each process is performed, image information generated by the camera 201, event information detected by the substrate processing device 2, and manipulation information of a user (an operator, a production manager, a maintenance manager, etc.) on the substrate processing device 2.

The database device 3 is an apparatus that manages history information 30 when the polishing process is performed in the substrate processing device 2. The database device 3 receives the various reports R from the substrate processing device 2 at any time and registers the reports R for each substrate processing device 2 in the history information 30, so that contents of the report R are accumulated in the history information 30 along with date and time information. In addition to the history information 30, the database device 3 may also store the device setting information 265 and the substrate recipe information 266. In that case, the substrate processing device 2 may refer to these information.

The machine-learning device 4 operates as a main configuration for the learning phase in the machine learning. For example, the machine-learning device 4 acquires, as the learning data 11, part of the history information 30 from the database device 3, and performs the machine learning to create a learning model 10 to be used in the information processing device 5. The learning model 10 as the learned model is provided to the information processing device 5 via the network 7, a storage medium, or the like. In this embodiment, supervised learning is employed as a method of the machine learning.

The information processing device 5 operates as a main configuration for an inference phase in the machine learning. When a crack (including damage, such as chipping or cracking) occurs in the wafer W during the substrate processing process performed by the substrate processing device 2, the information processing device 5 identifies a process that causes the crack in the wafer W (a crack occurrence process) using the learning model 10 generated by the machine-learning device 4, and transmits crack occurrence process information indicating the process that causes the crack in the wafer W to the database device 3, the user terminal device 6, etc.

The user terminal device 6 is a terminal device used by a user. The user terminal device 6 may be a stationary device or a portable device. The user terminal device 6 receives various input manipulations via a display screen of an application program, a web browser, etc., and displays various information (for example, event notification, the crack occurrence process information, the history information 30, etc.) via the display screen. The user terminal device 6 includes a camera 60 that is built-in or connectable to an external device.

For example, when a wafer W is cracked, the camera 201 of the substrate processing device 2 and the camera 60 of the user terminal device 6 function an imaging device that photographs the wafer W having a crack (hereinafter referred to as “cracked wafer”) and generates image information. The imaging device may be a device, such as the camera 201 of the substrate processing device 2, that automatically photographs the cracked wafer W during a series of operations of the substrate processing device 2, or may be a device, such as the camera 60 of the user terminal device 6, that manually photographs the cracked wafer W based on the user's photographing operation. The imaging device may be either the camera 201 of the substrate processing device 2 or the camera 60 of the user terminal device 6, or instead of or in addition to these cameras, the imaging device may be configured with an external device, such as an appearance inspection device. Furthermore, the camera 201 of the substrate processing device 2 may automatically photograph all wafers W, regardless of whether or not the wafers W are cracked.

### (Substrate Processing Device 2)

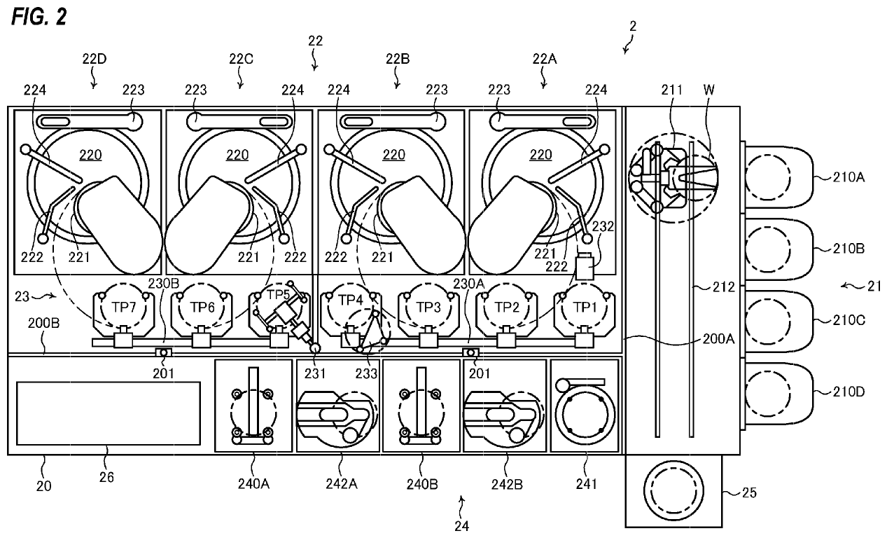

FIG. 2 is a plan view showing an example of the substrate processing device 2. The substrate processing device 2 includes a load-unload unit 21, a polishing unit 22, a substrate transport unit 23, a cleaning unit 24, a film-thickness measuring unit 25, and a control unit 26 which are arranged inside a housing 20 that is substantially rectangular in plan view. The load-unload unit 21 is isolated from the polishing unit 22, the substrate transport unit 23, and the cleaning unit 24 by a first partition wall 200A. The substrate transport unit 23 is isolated from the cleaning unit 24 by a second partition wall 200B.

### (Load-Unload Unit)

The load-unload unit 21 includes first to fourth front load sections 210A to 210D on which wafer cassettes (FOUPs, etc.), capable of storing a large number of wafers W along a vertical direction, are placed, a transfer robot 211 that is movable along the storage direction (vertical direction) of the wafers W in each wafer cassette, and a horizontally-moving mechanism 212 for moving the transfer robot 211 along an arrangement direction of the first to fourth front load sections 210A to 210D (i.e., along a direction of a shorter side of the housing 20).

The transfer robot 211 is configured to be accessible to the wafer cassette placed on each of the first to fourth front load sections 210A to 210D, the substrate transport unit 23 (specifically, a lifter 232, which will be described later), the cleaning unit 24 (specifically, a drying chamber 241, which will be described later), and the film-thickness measuring unit 25. The transfer robot 211 includes upper and lower hands (not shown) for transporting the wafer W between the wafer cassette, the substrate transport unit 23, the cleaning unit 24, and the film-thickness measuring unit 25. The lower hand is used when transporting the wafer W before processing of the wafer W, and the upper hand is used when transporting the wafer W after processing of the wafer W. When the wafer W is transported to and from the substrate transport unit 23 or the cleaning unit 24, a shutter (not shown) provided on the first partition wall 200A is opened and closed.

### (Polishing Unit)

The polishing unit 22 includes first to fourth polishing sections 22A to 22D each configured to perform the polishing process (planarization) on the wafer W. The first to fourth polishing sections 22A to 22D are arranged in parallel along the longitudinal direction of the housing 20.

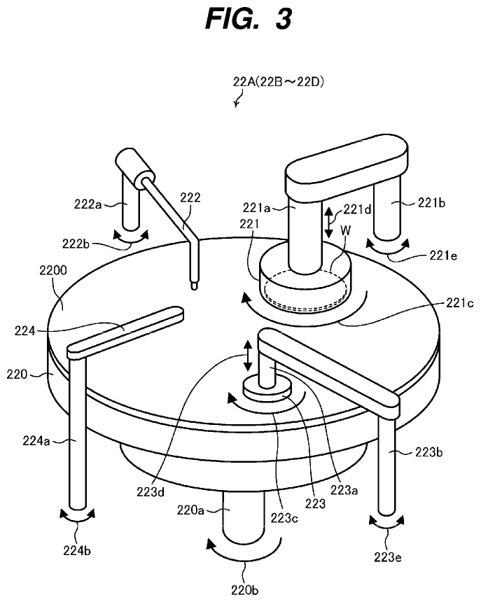

FIG. 3 is a perspective view showing an example of the first to fourth polishing sections 22A to 22D. The first to fourth polishing sections 22A to 22D have common basic configurations and functions.

Each of the first to fourth polishing units 22A to 22D includes a polishing table 220 to which a polishing pad 2200 having a polishing surface is attached, a top ring (polishing head) 221 for holding the wafer W and polishing the wafer W while pressing the wafer W against the polishing pad 2200 on the polishing table 220, a polishing-liquid supply nozzle 222 for supplying a polishing liquid (slurry) or a dressing liquid (for example, pure water) to the polishing pad 2200, a dresser 223 for dressing the polishing surface of the polishing pad 2200, and an atomizer 224 for atomizing a mixture of a liquid (for example, pure water) and a gas (for example, nitrogen gas) or atomizing a liquid (for example, pure water) and emitting the atomized fluid onto the polishing surface.

The polishing table 220 is supported by a polishing table shaft 220a. The polishing unit includes a rotating mechanism 220b that rotates the polishing table 220 an axis of the polishing table 220. The top ring 221 is supported by a top ring shaft 221a that is movable in the vertical direction. The polishing unit includes a rotating mechanism 221c that rotates the top ring 221 around an axis of the top ring 221, a vertical movement mechanism 221d that moves the top ring 221 in the vertical direction, and a rotation movement mechanism 221e that rotates (swings) the top ring 221 around the support shaft 221b. The polishing-liquid supply nozzle 222 is supported by a support shaft 222a. The polishing unit includes a rotation movement mechanism 222b that rotates and moves the polishing-liquid supply nozzle 222 around the support shaft 222a. The dresser 223 is supported by a dresser shaft 223a that is movable in the vertical direction. The polishing unit includes a rotating mechanism 223c that rotates the dresser 223 around an axis of the dresser 223, a vertical movement mechanism 223d that moves the dresser 223 in the vertical direction, and a rotation movement mechanism 223e that rotates the dresser 223 around the support shaft 223b. The atomizer 224 is supported by a support shaft 224a. The polishing unit includes a rotation movement mechanism 224b that rotates the atomizer 224 around the support shaft 224a.

In FIG. 3, specific configurations of the rotating mechanisms 220b, 221c, 223c, the vertical movement mechanisms 221d, 223d, and the rotational movement mechanisms 221e, 222b, 223e, 224b are omitted, but each mechanism may be constructed by appropriately combining actuator (e.g., motor, air cylinder), driving force transmission mechanism (e.g., linear guide, ball screw, gear, belt, coupling, bearing), and sensor (e.g., linear sensor, encoder sensor, limit sensor).

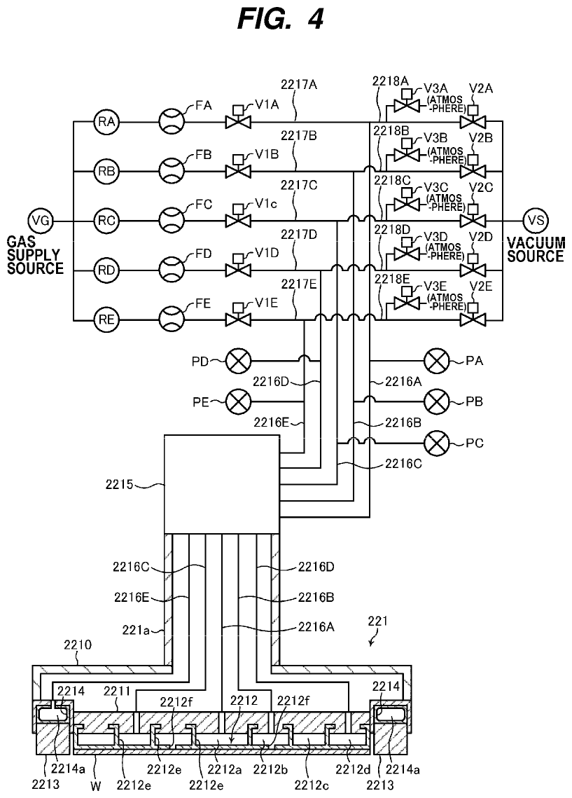

FIG. 4 is a cross-sectional view schematically showing an example of the top ring 221. The top ring 221 includes a top ring body 2210 attached to the top ring shaft 221a, a substantially disc-shaped carrier 2211 arranged inside the top ring body 2210, a membrane 2212 arranged beneath the carrier 2211 and presses the wafer W against the polishing pad 2200, a substantially annular retainer ring 2213 that is disposed at a periphery of the carrier 2211 and directly presses the polishing pad 2200, and a retainer ring airbag 2214 arranged between the top ring body 2210 and the retainer ring 2213 and presses the retainer ring 2213 against the polishing pad 2200.

The membrane 2212 is an elastic membrane, and has a plurality of concentric partition walls 2212e therein that form four membrane pressure chambers 2212a to 2212d arranged concentrically from the center toward the circumference of the top ring body 2210. Further, the membrane 2212 has a plurality of holes 2212f for adsorbing the wafer W on its lower surface, and functions as a substrate holding surface for holding the wafer W. The retaining ring airbag 2214 is formed of an elastic membrane and has a retaining-ring pressure chamber 2214a therein. The configurations of the top ring 221 may be changed as appropriate, and the top ring 221 may have a pressure chamber that presses the entire carrier 2211. The number and shape of the membrane pressure chambers of the membrane 2212 may be changed as appropriate. The number and arrangement of the suction holes 2212f may be changed as appropriate. Furthermore, the membrane 2212 may not have the suction holes 2212f.

First to fourth flow paths 2216A to 2216D are coupled to the first to fourth membrane pressure chambers 2212a to 2212d, respectively, and a fifth flow path 2216E is coupled to the retaining-ring pressure chamber 2214a. The first to fifth flow paths 2216A to 2216E communicate with an exterior via a rotary joint 2215 provided on the top ring shaft 221a. The first to fifth flow paths 2216A to 2216E are divided into first branch paths 2217A to 2217E and second branch paths 2218A to 2218E. Pressure sensors PA to PE are installed in the first to fifth flow paths 2216A to 2216E, respectively. The first branch paths 2217A to 2217E are coupled to a gas supply source GS of pressurized fluid (air, nitrogen, etc.) via valves V1A to V1E, flow-rate sensors FA to FE, and pressure regulators RA to RE. The second branch paths 2218A to 2218E are coupled to a vacuum source VS via valves V2A to V2E, respectively, and are configured to be able to communicate with the atmosphere via valves V3A to V3E.

The wafer W is held by suction on the lower surface of the top ring 221 and is moved to a predetermined polishing position above the polishing table 220 for polishing. Thereafter, the wafer W is polished by being pressed by the top ring 221 against the polishing surface of the polishing pad 2200 on which the polishing liquid is suppled from the polishing-liquid supply nozzle 222. At this time, the top ring 221 independently controls the pressure regulators RA to RE to generate pressing forces that press the wafer W against the polishing pad 2200 via the pressurized fluid supplied to the first to fourth membrane pressure chambers 2212a to 2212d while adjusting the pressing forces for respective regions of the wafer W. A pressing force for pressing the retainer ring 2213 against the polishing pad 2200 is adjusted by the pressurized fluid supplied to the retainer-ring pressure chamber 2214a. The pressures of the pressurized fluid supplied to the first to fourth membrane pressure chambers 2212a to 2212d and the retaining-ring pressure chamber 2214a are measured by the pressure sensors PA to PE, respectively, and the flow rates of the pressurized gas are measured by the flow-rate sensors FA to FE, respectively.

### (Substrate Transport Unit)

As shown in FIG. 2, the substrate transport unit 23 includes first and second linear transporters that are horizontally movable along the arrangement direction of the first to fourth polishing sections 22A to 22D (i.e., the longitudinal direction of the housing 20), a swing transporter 231 arranged between the first and second linear transporters 230A, 230B, a lifter 232 arranged near the load-unload unit 21, and a temporary station 233 for the wafer W arranged near the cleaning unit 24. Furthermore, cameras 201 are arranged at positions where the cameras 201 can photograph the wafers W being transported by the first and second linear transporters 230A and 230B.

The first linear transporter 230A is arranged adjacent to the first and second polishing sections 22A and 22B and is configured to transport the wafer W to four transport positions (which will be referred to as first to fourth transfer positions TP1 to TP4 in the order from the load-unload-unit-21-side). The second transfer position TP2 is a position where the wafer W is delivered to the first polishing section 22A. The top ring 221 of the first polishing section 22A is configured to be movable between the second transfer position TP2 and the polishing position by the oscillation motion of the top ring 221 of the first polishing section 22A. The third transfer position TP3 is a position where the wafer W is delivered to the second polishing section 22B. The top ring 221 of the second polishing section 22B is configured to be movable between the third transfer position TP3 and the polishing position by the oscillation motion of the top ring 221 of the second polishing section 22B.

The second linear transporter 230B is arranged adjacent to the third and fourth polishing sections 22C and 22D and is configured to transport the wafer W to three transport positions (which will be referred to as fifth to seventh transfer positions TP5 and TP7 in the order from the load-unload-unit-21-side). The sixth transfer position TP6 is a position where the wafer W is delivered to the third polishing section 22C. The top ring 221 of the third polishing section 22C is configured to be movable between the sixth transfer position TP6 and the polishing position by the oscillation motion of the top ring 221 of the third polishing section 22C. The seventh transfer position TP7 is a position where the wafer W is delivered to the fourth polishing section 22D. The top ring 221 of the fourth polishing section 22D is configured to be movable between the seventh transfer position TP7 and the polishing position by the oscillation motion of the top ring 221 of the fourth polishing section 22D.

The swing transporter 231 is disposed adjacent to the fourth and fifth transfer positions TP4 and TP5. The swing transporter 231 has a hand that is movable between the fourth and fifth transfer positions TP4 and TP5. The swing transporter 231 is configured to transport the wafer W between the first and second linear transporters 230A and 230B and place the wafer W temporarily on the temporary station 233.

The lifter 232 is disposed adjacent to the first transfer position TP1. The lifter 232 is configured to transport the wafer W between the first transfer position TP1 and the transfer robot 211 of the load-unload unit 21. When the wafer W is transported, the shutter (not shown) provided on the first partition wall 200A is opened and closed.

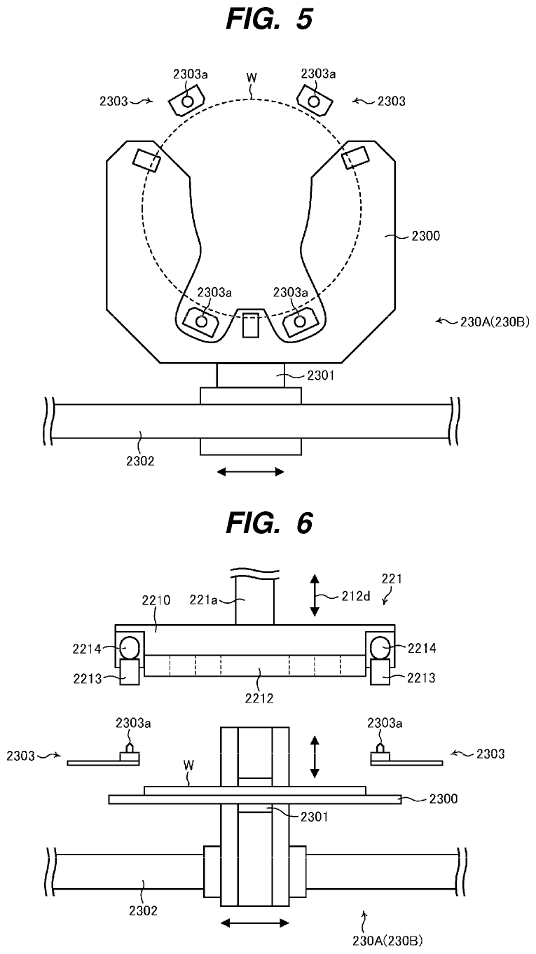

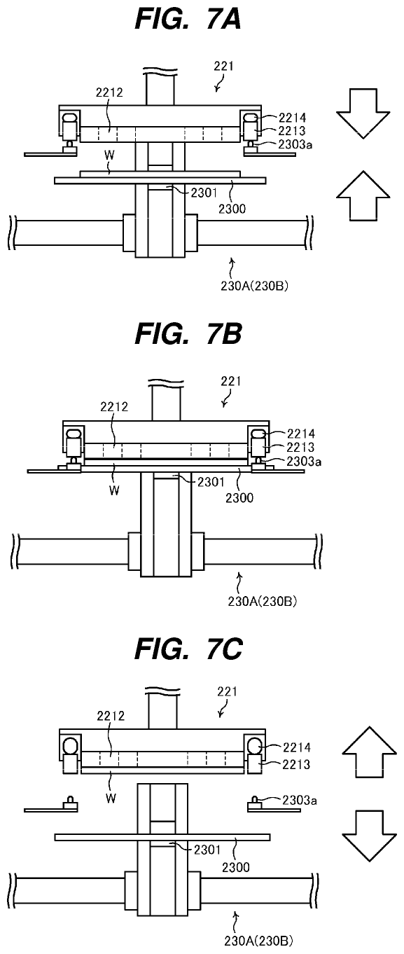

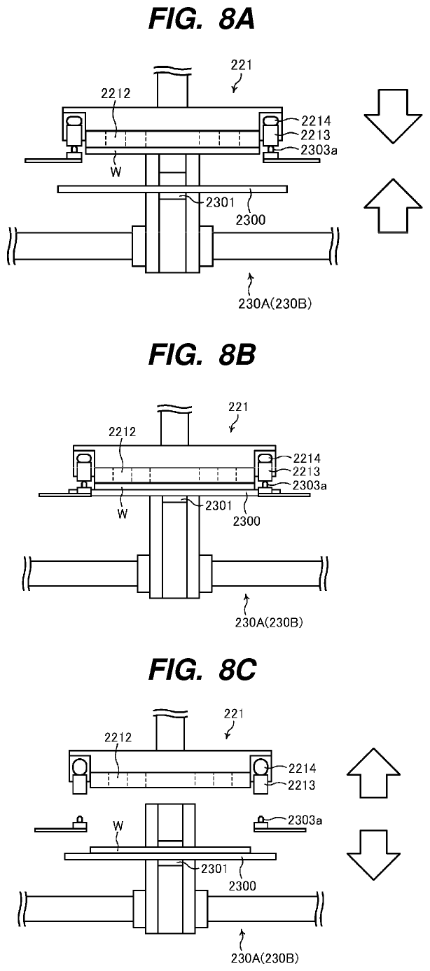

FIG. 5 is a plan view schematically showing an example of the first and second linear transporters 230A and 230B. FIG. 6 is a front view schematically showing an example of the first and second linear transporters 230A and 230B. FIG. 7 is a schematic diagram showing an example of a substrate receiving process. FIG. 8 is a schematic diagram showing an example of a substrate delivery process.

Each of the first and second linear transporters 230A, 230B includes a transport hand 2300 for holding the wafer W, a vertical movement mechanism 2301 configured to move the transport hand 2300 along the vertical direction, a horizontal movement mechanism 2302 configured to move the transport hand 2300 and the vertical movement mechanism 2301 along the arrangement direction of the first to fourth polishing sections 22A to 22D (i.e., the longitudinal direction of the housing 20), and retainer-ring stations 2303 arranged at positions (the second transfer position TP2, the third transfer position TP3, the sixth transfer position TP6, and the seventh transfer position TP7) where the wafer W is transferred to and from the top ring 221. The first and second linear transporters 230A and 230B include a plurality of sets of the transport hand 2300, the vertical movement mechanism 2301, and the horizontal movement mechanism 2302.

The transport hand 2300 has a shape configured to support a periphery of a lower surface of the wafer W. The retainer-ring station 2303 includes push-up pins 2303a that are disposed at positions facing the retainer ring 2213 of the top ring 221 and push up the retainer ring 2213. The retainer-ring station 2303 is installed at a position where the retainer-ring station 2303 does not interfere with the transport hand 2300 when the transport hand 2300 is positioned below the retainer-ring station 2303 by the horizontal movement mechanism 2302 and raised by the vertical movement mechanism 2301. The retainer-ring station 2303 may include a release nozzle configured to supply fluid for releasing the wafer W.

Although the specific configurations of the vertical movement mechanism 2301 and the horizontal movement mechanism 2302 are omitted in FIGS. 5, 6, the vertical movement mechanism 2301 and the horizontal movement mechanism 2302 may be constructed by appropriately combining actuator (e.g., motor, air cylinder), driving force transmission mechanism (e.g., linear guide, ball screw, gear, belt, coupling, bearing), and sensor (e.g., linear sensor, encoder sensor, limit sensor).

FIG. 7 illustrates the substrate receiving process in which the polishing unit 22 receives a wafer W before polishing from the substrate transport unit 23. Specifically, the top ring 221 that is not holding a wafer W is lowered, while the transport hand 2300 holding a wafer W is elevated. As the top ring 221 is lowered, the retainer ring 2213 is pushed up by the push-up pins 801. When the transport hand 2300 is further elevated, the upper surface of the wafer W comes into contact with the lower surface of the membrane 2212. At this time, for example, the second membrane pressure chamber 2212b corresponding to the position where the suction holes 2212f are formed is evacuated by the vacuum source VS, so that the wafer W is attracted and held on the membrane 2212. Then, the top ring 221 that holds the wafer W is elevated, and the transport hand 2300 that that delivered the wafer W is lowered.

FIG. 8 illustrates the substrate delivery process in which the polishing unit 22 delivers a wafer W after polishing to the substrate transport unit 23. Specifically, the top ring 221 holding a wafer W by the vacuum suction is lowered, while the transport hand 2300 that does not hold a wafer W is elevated. As the top ring 221 is lowered, the retainer ring 2213 is pushed up by the push-up pins 801. When the transport hand 2300 is further elevated, the lower surface of the wafer W approaches the transport hand 2300. At this time, for example, the vacuum evacuation of the second membrane pressure chamber 2212b corresponding to the position where the suction holes 2212f are formed is stopped, and the third membrane pressure chamber 2212c located outwardly of the second membrane pressure chamber 2212b is supplied with the pressurized fluid, so that the wafer W is released from the membrane 2212. Then, the top ring 221 that has released the wafer W is raised, and the transport hand 2300 that has received the wafer W is lowered.

### (Cleaning Unit)

As shown in FIG. 2, the cleaning unit 24 includes first and second cleaning chambers 240A and 240B for cleaning the wafer W using cleaning liquid, a drying chamber 241 for drying the wafer W, and first and second transporting chambers 242A and 242B for transporting the wafer W. These chambers of the cleaning unit 24 are partitioned and arranged along the first and second linear transporters 230A and 230B. For example, the chambers of the cleaning unit 24 are arranged in the order of the first cleaning chamber 240A, the first transporting chamber 242A, the second cleaning chamber 240B, the first transporting chamber 242B, and the drying chamber 241 (in the order of distance from the load-unload unit 21).

### (Film-Thickness Measuring Unit)

The film-thickness measuring unit 25 is a measuring device that measures the film thickness of the wafer W before or after the polishing process. The film-thickness measuring unit 25 is, for example, an optical film-thickness measuring device, an eddy current type film-thickness measuring device, or the like. The transfer robot 211 transports the wafer W to and from each film-thickness measuring module.

### (Control Unit)

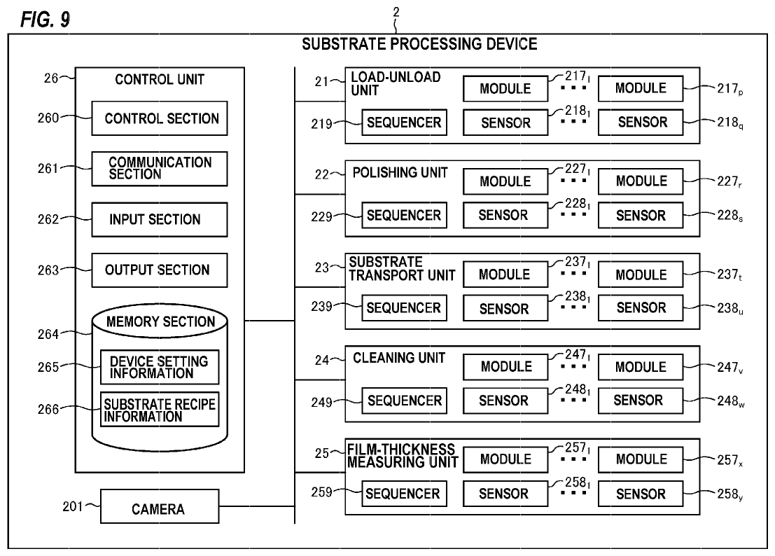

FIG. 9 is a block diagram showing an example of the substrate processing device 2. The control unit 26 is electrically coupled to each of the units 21 to 25 and the camera 201, and functions as a control section that comprehensively controls the units 21 to 25 and the camera 201.

The load-unload unit 21 includes modules 2171 to 217p (for example, the transfer robot 211, etc.) composed of various actuators, sensors 2181 to 218q arranged in the modules 2171 to 217p, respectively, for detecting data (detection values) necessary for controlling the modules 2171 to 217p, and a sequencer 219 for controlling the operations of the modules 2171 to 217p based on the detection values of the sensors 2181 to 218q.

The polishing unit 22 includes modules 2271 to 227r (for example, the polishing table 220, the top ring 221, the polishing-liquid supply nozzle 222, the dresser 223, the atomizer 224, etc.) composed of various actuators, sensors 2281 to 228s arranged in the modules 2271 to 227r, respectively, for detecting data (detection values) necessary for controlling the modules 2271 to 227r, and a sequencer 229 for controlling the operations of the modules 2271 to 227r based on the detection values of the sensors 2281 to 228s.

Examples of the sensors 2281 to 228s of the polishing unit 22 include a sensor for detecting a rotation speed of the polishing table 220, a sensor for detecting a rotation torque of the polishing table 220, a sensor for detecting a rotation speed of the top ring 221, a sensor for detecting a rotation torque of the top ring 221, a sensor for detecting an oscillation position of the top ring 221, a sensor for detecting an oscillation torque of the top ring 221, a sensor for detecting a height of the top ring 221, and a sensor for detecting an elevating torque of the top ring 221, sensors for detecting pressures (positive pressure and negative pressure) in the first to fourth membrane pressure chambers 2212a to 2212d and the retaining-ring pressure chamber 2214a, sensors for detecting flow rates of the pressurized fluid supplied to the first to fourth membrane pressure chambers 2212a to 2212d and the retaining-ring pressure chamber 2214a, a sensor for detecting a flow rate of the polishing liquid supplied from the polishing-liquid supply nozzle 222, a sensor for detecting a dropping position of the polishing-liquid supply nozzle 222, etc.

The substrate transport unit 23 includes modules 2371 to 237t (for example, the first and second linear transporters 230A, 230B, the swing transporter 231, the lifter 232, etc.) configured with various actuators, sensors 2381 to 238u arranged in the modules 2371 to 237t, respectively, for detecting data (detection values) necessary for controlling the modules 2371 to 237t, and a sequencer 239 for controlling the operations of the modules 2371 to 237t based on the detection values of the sensors 2381 to 238u.

Examples of the sensors 2381 to 238u of the substrate transport unit 23 include a sensor for detecting a position of the transport hand 2300, a sensor for detecting a height of the transport hand 2300, a sensor for detecting the presence or absence of a wafer W on the transport hand 2300, etc.

The cleaning unit 24 includes modules 2471 to 247v (for example, the first cleaning chamber 240A, the second cleaning chamber 240B, the drying chamber 241, etc.) configured with various actuators, sensors 2481 to 248w arranged in the modules 2471 to 247v, respectively, for detecting data (detection values) necessary for controlling the modules 2471 to 247v, and a sequencer 249 for controlling the operations of the modules 2471 to 247v based on the detection values of the sensors 2481 to 248w.

The film-thickness measuring unit 25 includes modules 2571 to 257x (for example, film-thickness measuring module, etc.) configured with various actuators, sensors 2581 to 258y arranged in the modules 2571 to 257x, respectively, for detecting data (detection values) necessary for controlling the modules 2571 to 257x, and a sequencer 259 for controlling the operations of the modules 2571 to 257x based on the detection values of the sensor 2581 to 258y.

The control unit 26 includes a control section 260, a communication section 261, an input section 262, an output section 263, and a memory section 264. The control unit 26 is comprised of, for example, a general-purpose or dedicated computer (see FIG. 11, which will be described later).

The communication section 261 is coupled to the network 7 and functions as a communication interface for transmitting and receiving various data. The input section 262 receives various input operations. The output section 263 functions as a user interface by outputting various information via a display screen, lighting of signal tower, or buzzer sound.

The memory section 264 stores therein various programs (operating system (OS), application programs, web browser, etc.) and data (the device setting information 265, the substrate recipe information 266, etc.) used in the operations of the substrate processing device 2. The device setting information 265 and the substrate recipe information 266 are data that can be edited by the user via the display screen.

The control section 260 obtains detection values of the multiple sensors 2181 to 218q, 2281 to 228s, 2381 to 238u, 2481 to 248w, 2581 to 258y (hereinafter referred to as “sensor group”). The control section 260 operates the multiple modules 2171 to 217p, 2271 to 227r, 2371 to 237t, 2471 to 247v, and 2571 to 257x (hereinafter referred to as “module group”) in cooperation to perform a series of processes including loading, polishing, cleaning, drying, film thickness measuring, and unloading.

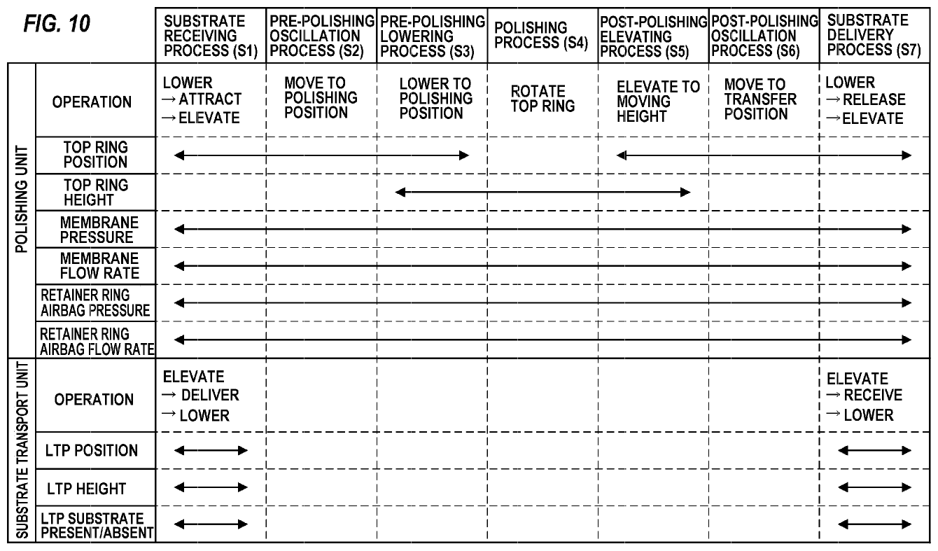

FIG. 10 is a timing chart showing an example of a substrate processing process performed by the substrate processing device 2. The substrate processing process shown in FIG. 10 includes a part of the series of processes described above. Specifically, substrate processing process includes transferring a wafer W before polishing from the first or second linear transporter 230A, 230B of the substrate transport unit 23 the top ring 221 of the polishing unit 22, polishing the wafer W in the polishing unit 22, and transferring the wafer W after polishing to the first or second linear transporter 230A or 230B.

The substrate processing process includes a substrate receiving process S1 (FIG. 7) in which the top ring 221 receives the wafer W from the first or second linear transporter 230A, 230B before the polishing process, a pre-polishing oscillation process S2 in which the top ring 221 moves the wafer W to the polishing position above the polishing table 220 before the polishing process, a pre-polishing lowering process S3 in which the top ring 221 lowers the wafer W to the polishing height before the polishing process, a polishing process S4 in which the top ring 221 performs the polishing process on the wafer W, a post-polishing elevating process S5 in which the top ring 221 elevates the wafer W to a moving height after the polishing process, a post-polishing oscillation process S6 in which the top ring 221 moves the wafer W to the transfer position on the retainer ring station 2303 after the polishing process, and a substrate delivery process S7 (FIG. 8) in which the top ring 221 delivers the wafer W to the first or second linear transporter 230A, 230B after the polishing process.

In this embodiment, the above-mentioned substrate processing process is performed between the first and second polishing sections 22A, 22B and the first linear transporter 230A, and the above-mentioned substrate processing process is performed between the third and fourth polishing sections 22C, 22D and the second linear transporter 230B.

### (Hardware Configuration of Each Device)

FIG. 11 is a hardware configuration diagram showing an example of a computer 900. Each of the control unit 26, the database device 3, the machine-learning device 4, the information processing device 5, and the user terminal device 6 of the substrate processing device 2 is configured by the general-purpose or dedicated computer 900.

As shown in FIG. 11, main components of the computer 900 include buses 910, a processor 912, a memory 914, an input device 916, an output device 917, a display device 918, a storage device 920, a communication I/F (interface) section 922, an external device I/F section 924, an I/O (input/output) device I/F section 926, and a media input/output section 928. The above components may be omitted as appropriate depending on an application in which the computer 900 is used.

The processor 912 includes one or more arithmetic processing unit(s) (CPU (Central Processing Unit), MPU (Micro-processing unit), DSP (digital signal processor), GPU (Graphics Processing Unit), etc.), and operates as a controller configured to control the entire computer 900. The memory 914 stores various data and programs 930, and includes, for example, a volatile memory (DRAM, SRAM, etc.) that functions as a main memory, a non-volatile memory (ROM), a flash memory, etc.

The input device 916 includes, for example, a keyboard, a mouse, a numeric keypad, an electronic pen, etc., and functions as an input section. The output device 917 includes, for example, a sound (voice) output device, a vibration device, etc., and functions as an output section. The display device 918 includes, for example, a liquid crystal display, an organic EL display, electronic paper, a projector, etc., and functions as an output section. The input device 916 and the display device 918 may be configured integrally, such as a touch panel display. The storage device 920 includes, for example, HDD (Hard Disk Drive), SSD (Solid State Drive), etc., and functions as a storage section. The storage device 920 stores various data necessary for executing the operating system and the programs 930.

The communication I/F section 922 is coupled to a network 940, such as the Internet or an intranet (which may be the same as the network 7 in FIG. 1), in a wired manner or a wireless manner, and transmits and receives data to and from another computer according to a predetermined communication standard. The communication I/F section 922 functions as a communication unit that sends and receives information. The external device I/F section 924 is coupled to an external device 950, such as camera, printer, scanner, reader/writer, etc. in a wired manner or a wireless manner, and serves as a communication section that transmits and receives data to and from the external device 950 according to a predetermined communication standard. The I/O device I/F unit 926 is coupled to I/O device 960, such as various sensors or actuators, and functions as a communication unit that transmits and receives various signals, such as detection signals from the sensors or control signals to the actuators, and data to and from the I/O device 960. The media input/output unit 928 is constituted of a drive device, such as a DVD drive or a CD drive, and writes and reads data into and from medium (non-transitory storage medium) 970, such as a DVD or a CD.

In the computer 900 having the above configurations, the processor 912 calls the program 930 stored in the storage device 920 into the memory 914 and executes the program 930, and controls each part of the computer 900 via the buses 910. The program 930 may be stored in the memory 914 instead of the storage device 920. The program 930 may be stored in the medium 970 in an installable file format or an executable file format, and may be provided to the computer 900 via the media input/output unit 928. The program 930 may be provided to the computer 900 by being downloaded via the network 940 and the communication I/F unit 922. The computer 900 performs various functions realized by the processor 912 executing the programs 930. The computer 900 may include hardware, such an FPGA, an ASIC, etc. for executing the above-described various functions.

The computer 900 is, for example, a stationary computer or a portable computer, and is an electronic device in arbitrary form. The computer 900 may be a client computer, a server computer, or a cloud computer. The computer 900 may be applied to devices other than the devices 2 to 6.

### (History Information 30)

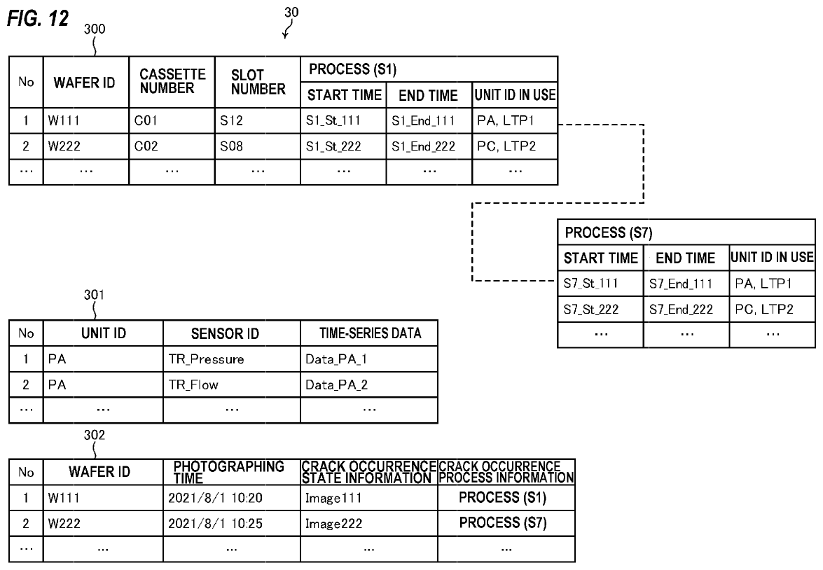

FIG. 12 is a data configuration diagram showing an example of history information 30 managed by the database device 3. The history information 30 includes tables in which the various reports R from the substrate processing device 2 are classified and registered. Specifically, the tables include a process history table 300 regarding process information, device-state history table 301 regarding device-state information (detection values of the sensor, command values to actuators etc.), and a crack occurrence history table 302 regarding image information and sketch information described later. In addition to the above tables, the history information 30 includes an event history table regarding event information, an operation history table regarding operation information, etc., but detailed descriptions thereof are omitted.

For example, a wafer ID, cassette number, slot number, start time of each process S1 to S7, end time of each process S1 to S7, ID of the unit used in each process S1 to S7, etc. are registered in each record of the process history table 300. Information regarding process other than the processes S1 to S7 may be registered in the process history table 300.

For example, a unit ID, a sensor ID (or actuator ID), time-series data, etc. are registered in each record of the device-state history table 301. The time-series data are the detection values of the sensor (or command values to an actuator) sampled at predetermined time intervals.

For example, a wafer ID, acquisition time, crack occurrence state information, crack occurrence process information, etc. are registered in each record of the crack occurrence history table 302. The crack occurrence state information includes the image information of the cracked wafer W generated by either the camera 201 of the substrate processing device 2 or the camera 60 of the user terminal device 6, or the sketch information of the cracked wafer W sketched by the user. The crack occurrence process information includes a crack occurrence process identified by the user or the information processing device 5.

By referring to the process history table 300 and the device-state history table 301, the time-series data of each sensor (or time-series data of each actuator) can be extracted as the device-state information when each process S1 to S7 included in the substrate processing process is performed on the wafer W identified by the wafer ID. Furthermore, the crack occurrence state information and the crack occurrence process information for the wafer W identified by the wafer ID can be extracted by further referring to the crack occurrence history table 302.

### (Machine-Learning Device 4)

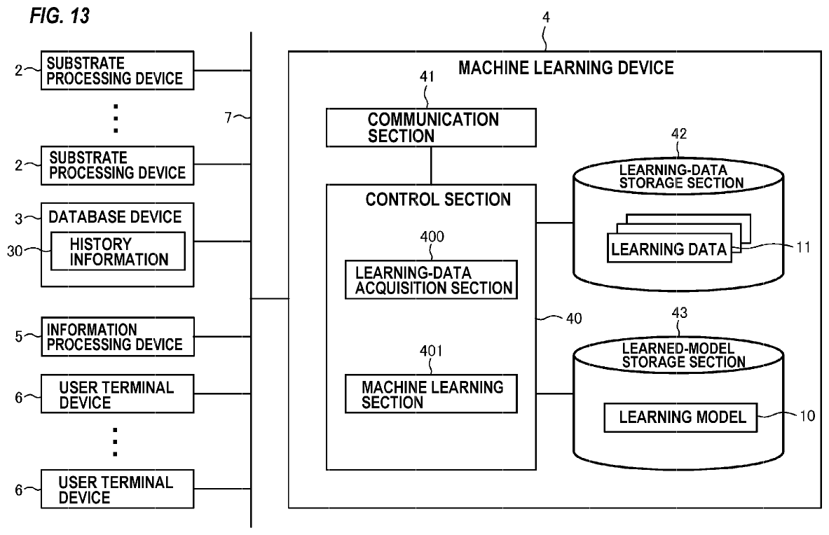

FIG. 13 is a block diagram showing an example of the machine-learning device 4. The machine-learning device 4 includes a control section 40, a communication section 41, a learning-data storage section 42, and a learned-model storage section 43.

The control section 40 functions as a learning-data acquisition section 400 and a machine-learning section 401. The communication section 41 is coupled to external devices (for example, the substrate processing device 2, the database device 3, the information processing device 5, the user terminal device 6, etc.) via the network 7. The communication section 41 serves as a communication interface for transmitting and receiving various data.

The learning-data acquisition section 400 is coupled to an external device via the communication section 41 and the network 7. The learning-data acquisition section 400 acquires the learning data 11 including the crack occurrence state information as input data and the crack occurrence process information as output data. The learning data 11 is data used as teaching data (or training data), verification data, and test data in supervised learning. The crack occurrence process information is used as ground-truth label or correct label in supervised learning.

The learning-data storage section 42 is a database that stores multiple sets of learning data 11 acquired by the learning-data acquisition section 400. The specific configuration of the database that constitutes the learning-data storage section 42 may be designed as appropriate.

The machine-learning section 401 performs the machine learning using the multiple sets of learning data 11 stored in the learning-data storage section 42. Specifically, the machine-learning section 401 inputs the multiple sets of learning data 11 to the learning model 10 and causes the learning model 10 to learn the correlation between the crack occurrence state information and the crack occurrence process information included in the learning data 11 to thereby create the learning model 10 as a learned model.

The learned-model storage section 43 is a database that stores the learning model 10 as a learned model (i.e., adjusted weight parameter group) created by the machine-learning section 401. The learning model 10 as the learned model stored in the learned-model storage section 43 is provided to a real system (for example, the information processing device 5) via the network 7, a storage medium, or the like. Although the learning-data storage section 42 and the learned-model storage section 43 are shown as separate storage sections in FIG. 13, they may be configured as a single storage section.

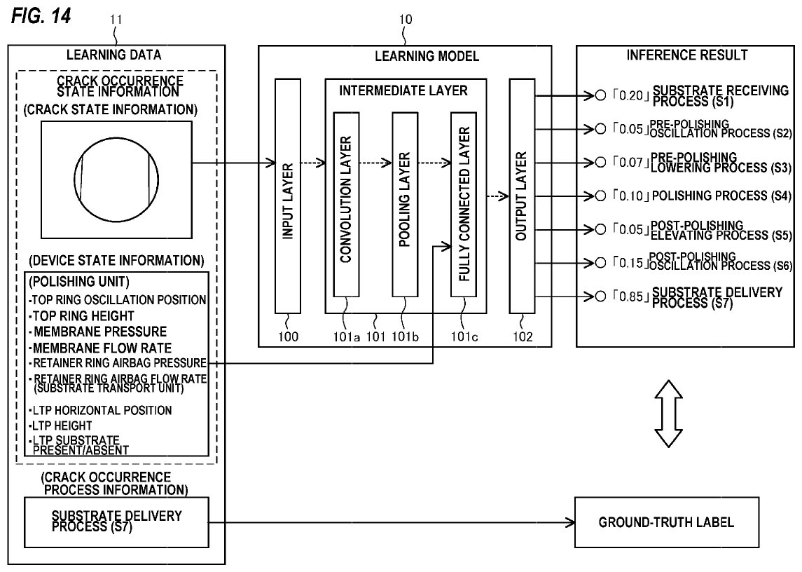

FIG. 14 is a diagram showing an example of the learning model 10 and the learning data 11. The learning data 11 used for the machine learning for the learning model 10 includes the crack occurrence state information including the crack state information and the device state information, and the crack occurrence process information.

The crack state information included in the crack occurrence state information is information indicating a crack state when a crack occurs in the wafer W in the substrate processing process performed by the substrate processing device 2. The crack state information is image information in which the cracked wafer W is photographed, or sketch information in which the cracked wafer W is sketched.

The image information is generated by the substrate processing device 2 (camera 201) or the user terminal device 6 (camera 60) as a result of photographing the cracked wafer W in a plan view. For example, in the case where the wafer W is broken into pieces, it is preferable that the cracked wafer W be photographed after the pieces are arranged into an original form of the wafer W so that the crack(s) can be seen. The image information may be either a monochrome image or a color image, or a two-dimensional image or a three-dimensional image.

The sketch information is, for example, a drawing of a crack and an outline of the wafer W having a notch when the wafer W is cracked. The sketch information may be generated, for example, by the user making a drawing line representing the cracked state of the wafer W using a drawing application program of the user terminal device 6, or by the user handwriting a drawing line representing the cracked state of the wafer W on a paper and then reading the paper using an image reading device, such as a scanner.

The device state information included in the crack occurrence state information is information indicating the state of the polishing unit 22 when the substrate processing process is performed on the cracked wafer W.

The device state information includes, as the state of the polishing unit 22, at least one of an oscillation position of the top ring 221, a height of the top ring 221, pressures in the first to fourth membrane pressure chambers 2212a to 2212 (membrane pressures), flow rates of the pressurized fluid supplied to the first to fourth membrane pressure chambers 2212a to 2212d (membrane flow rate), pressure in the retaining-ring pressure chamber 2214a (retaining-ring airbag pressure), and a flow rate of the pressurized fluid supplied to the retaining-ring pressure chamber 2214a (retaining-ring airbag flow rate). Note that the pressures in the first to fourth membrane pressure chambers 2212a to 2212d and the retaining-ring pressure chamber 2214a include pressures when the pressurized fluid is supplied, pressures when the pressurized fluid is released to the atmosphere, and pressures when the chambers are evacuated.

The device state information may include information indicating the state of the substrate transport unit 23 in addition to the state of the polishing unit 22. In that case, the device state information includes, as the state of the substrate transport unit 23, at least one of the position of the transport hand 2300 (LTP horizontal position), the height of the transport hand 2300 (LTP height), and the presence or absence of the wafer W on the transport hand 2300 (LTP substrate presence/absence).

The crack occurrence process information is information indicating a process (crack occurrence process) that causes a crack in the wafer W among the processes S1 to S7 included in the substrate processing process. The processes S1 to S7 included in the substrate processing process include, as shown in FIG. 10, the substrate receiving process S1, the pre-polishing oscillation process S2, the pre-polishing lowering process S3, the polishing process S4, the post-polishing elevating process S5, the post-polishing oscillation process S6, and the substrate delivery process S7.

The learning-data acquisition section 400 acquires the learning data 11 by referring to the history information 30 and receiving, as necessary, the input manipulations of the user from the user terminal device 6.

For example, when a wafer W is cracked, the learning-data acquisition section 400 refers to the process history table 300 and the device-state history table 301 of the history information 30 using the wafer ID that identifies the cracked wafer W, so that the learning-data acquisition section 400 can acquire, as device state information, the time-series data of the sensor group when the substrate processing process was performed on the cracked wafer W. Furthermore, the learning-data acquisition section 400 acquires the crack state information for the cracked wafer W by referring to the crack occurrence history table 302 of the history information 30 using the wafer ID. Instead of acquiring the crack state information from the history information 30, the learning-data acquisition section 400 may acquire the image information generated by the user's photographing operation or the sketch information generated by the user's drawing operation or the reading operation.

The user may analyze the state of the polishing unit 22 and the substrate transport unit 23 based on the way of cracking of the wafer W (features including direction of a crack and the number of cracks, etc.) and the device state information. The learning-data acquisition section 400 receives, as a result of the user's analysis, a user's designation operation that identifies a crack occurrence process for the cracked wafer W to thereby acquire the crack occurrence process information. Instead of receiving the user's designation operation, the learning-data acquisition section 400 may acquire the crack occurrence process information by referring to the crack occurrence history table 302 of the history information 30 using the wafer ID that identifies the cracked wafer W, if the crack occurrence process information as a result of the user's analysis performed in advance has already been registered in the crack occurrence history table 302 of the history information 30.

The learning model 10 employs, for example, a convolutional neural network (CNN) structure, and includes an input layer 100, intermediate layers 101, and an output layer 102. Synapses (not shown) connecting neurons are placed between the layers, and each synapse is associated with a weight. A weight parameter group including weights of the synapses is adjusted by the machine learning.

The input layer 100 has neurons corresponding to pixels of the crack occurrence state information (the image information or the sketch information) as the input data, and a pixel value of each pixel is input to each neuron. The intermediate layer 101 includes, for example, a convolution layer 101a, a pooling layer 101b, and a fully connected layer 101c. The fully connected layer 101c has multiple inputs not only for the pooling layer 101b but also for the device state information as input data. Feature values of the image information from the pooling layer 101b and variables indicating the device state information (e.g., time-series data of the sensor group) are input to the neurons of the fully connected layer 101c. The output layer 102 has neurons corresponding to the crack occurrence process (the processes S1 to S7) in the crack occurrence process information as output data. The output layer 102 outputs a determination result (inference result) for each process S1 to S7 as the output data. Specifically, the learning model 10 is a multi-class classification model, and outputs scores (reliabilities) when classified into the processes S1 to S7 with numerical values within a predetermined range (for example, 0 to 1).

In this embodiment, the device state information is acquired as the time-series data of the sensor group as shown in FIG. 14. The device state information may be changed as appropriate according to constructions of the polishing unit 22 (particularly the top ring 221) or the substrate transport unit 23. In addition, the device state information may include a command value to an actuator, or a parameter converted from a detection value of the sensor or a command value to an actuator, or a parameter calculated based on detection values of the sensors. As described above, when the definition of device state information is changed, data structure of the input data in the learning model 10 and the learning data 11 may be changed as appropriate.

Furthermore, in this embodiment, a case will be described where the substrate processing process by the substrate processing device 2 is divided into seven processes S1 to S7 as shown in FIGS. 10 and 14, but the positions at which the substrate processing processes are divided may be changed as appropriate. It may be divided into smaller sections or more broadly. Further, the substrate processing process may further include a step before the substrate receiving step S1, or may further include a step after the substrate delivery step S7. The polishing process may be performed in two stages, or three or more stages. For example, a wafer is polished in the first polishing section 22A and then polished in the second polishing section 22B. In another example, a wafer is polished in the third polishing section 22C and then polished in the fourth polishing section 22D. In these cases, the substrate processing process may include a series of these processes, i.e., multi-stage substrate processing processes. When the definition of the substrate processing process is changed as described above, the data structure of the output data in the learning model 10 and the learning data 11 may be changed as appropriate.

### (Machine-Learning Method)

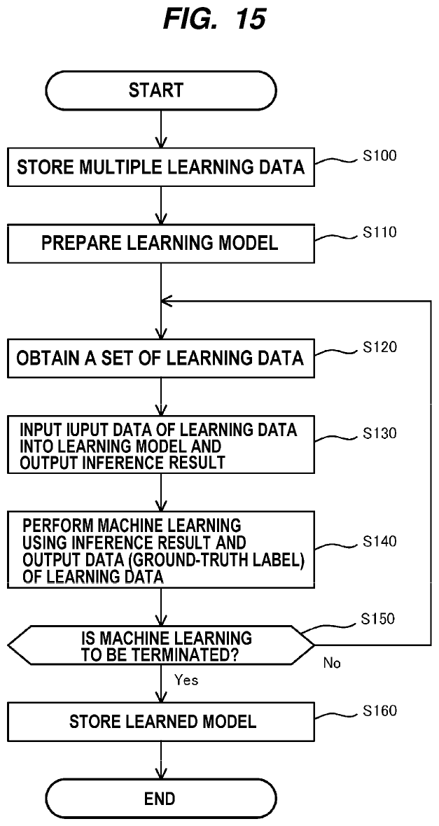

FIG. 15 is a flowchart showing an example of a machine-learning method performed by the machine-learning device 4.

First, in step S100, the learning-data acquisition section 400 obtains, from the history information 30, a desired number of learning data 11 as advance preparation for starting the machine learning, and stores the obtained learning data 11 in the learning-data storage section 42. The number of learning data 11 to be prepared may be set in consideration of the inference accuracy required for the learning model 10 finally obtained.

Next, in step S110, the machine-learning section 401 prepares the learning model 10 before learning for starting the machine learning. The learning model 10 prepared before learning in this embodiment is composed of the neural network model illustrated in FIG. 12, and the weight of each synapse is set to an initial value.

Next, in step S120, the machine-learning section 401 randomly obtains, for example, one set of learning data 11 from the multiple sets of learning data 11 stored in the learning-data storage section 42.

Next, in step S130, the machine-learning section 401 inputs the crack occurrence state information (the input data) included in the one set of learning data 11 to the input layer of the prepared learning model 10 before learning (or during learning). As a result, the crack occurrence process information (the output data) is output as the inference result from the output layer of the learning model 10. However, the output data is generated by the learning model 10 before learning (or during learning). Therefore, in the state before learning (or during learning), the output data as the inference result may indicate different information from the crack occurrence process information (ground-truth label) included in the learning data 11.

Next, in step S140, the machine-learning section 401 performs the machine learning by comparing the crack occurrence process information (ground-truth label) included in the one set of learning data 11 acquired in the step S120 with the crack occurrence process information (the output data) as the inference result output from the output layer in the step S130, and adjusting the weight wi of each synapse (backpropagation). In this way, the machine-learning section 401 causes the learning model 10 to learn the correlation between the crack occurrence state information and the crack occurrence process information.

Next, in step S150, the machine-learning section 401 determines whether or not a predetermined learning end condition is satisfied. For example, this determination is made based on an evaluation value of an error function based on the crack occurrence process information (ground-truth label) included in the learning data and the crack occurrence process information (the output data) output as the inference result, or based on the remaining number of unlearned learning data stored in the learning-data storage section 42.

In step S150, if the machine-learning section 401 has determined that the learning end condition is not satisfied and the machine learning is to be continued (No in step S150), the process returns to the step S120, and the steps S120 to S140 are performed on the learning model 10 multiple times using the unlearned learning data 11. On the other hand, in step S150, if the machine-learning section 401 has determined that the learning end condition is satisfied and the machine learning is to be terminated (Yes in step S150), the process proceeds to step S160.

Then, in step S160, the machine-learning section 401 stores, in the learned-model storage section 43, the learning model 10 as the learned model (adjusted weight parameter group) generated by adjusting the weight associated with each synapse. The sequence of machine-learning processes shown in FIG. 15 is completed. In the machine-learning method, the step S100 corresponds to a learning-data storing process, the steps S110 to S150 correspond to a machine-learning process, and the step S160 corresponds to a learned-model storing process.

As described above, the information processing device 5 and the information processing method according to the present embodiment can provide the learning model 10 that can identify (infer) the cause of crack in the wafer W from the crack occurrence state information including the crack state information and the device state information obtained in response to the occurrence of the crack in the wafer W.

### (Information Processing Device 5)

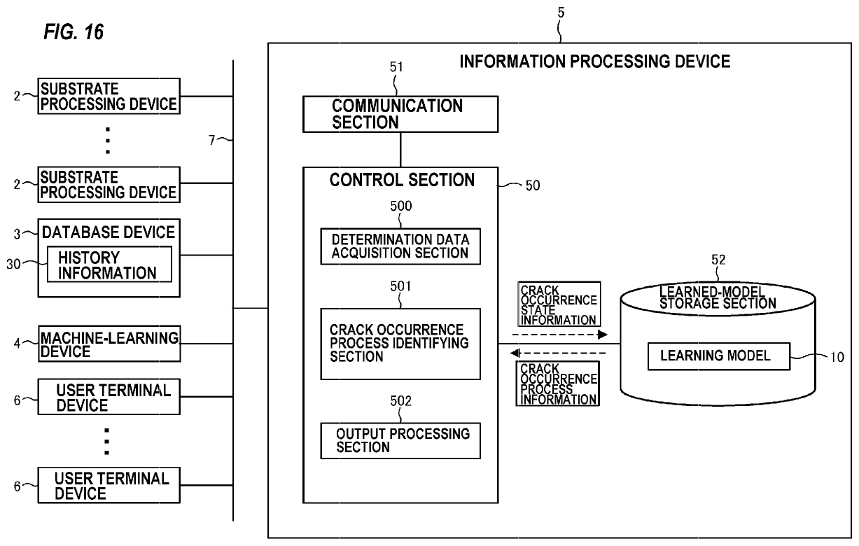

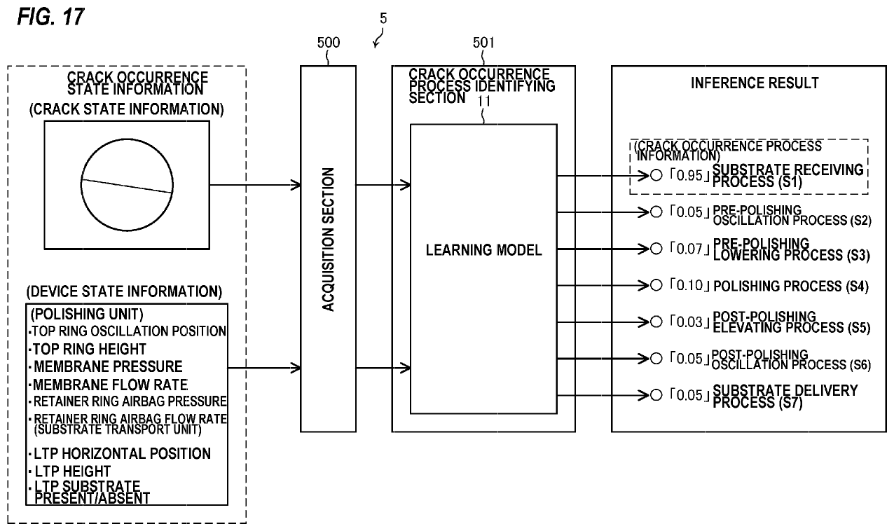

FIG. 16 is a block diagram showing an example of the information processing device 5. FIG. 17 is a functional explanatory diagram showing an example of the information processing device 5. The information processing device 5 includes a control section 50, a communication section 51, and a learned-model storage section 52.

The control section 50 functions as an information acquisition section 500, a crack occurrence process identifying section 501, and an output processing section 502. The communication section 51 is coupled to external devices (for example, the substrate processing device 2, the database device 3, the machine-learning device 4, the user terminal device 6, etc.) via the network 7, and serves as a communication interface for transmitting and receiving various data.

The information acquisition section 500 is coupled to an external device via the communication section 51 and the network 7 and acquires the crack occurrence state information including the crack state information and the device state information. For example, when the information acquisition section 500 receives a wafer ID identifying a cracked wafer W from the user terminal device 6, the information acquisition section 500 uses the wafer ID to refer to the process history table 300 and the device-state history table 301 of the history information 30 so that the information acquisition section 500 acquires, as the device state information, the time-series data of the sensor group when the substrate processing process is performed on the cracked wafer W. Furthermore, the information acquisition section 500 acquires the crack state information for the cracked wafer W by referring to the crack occurrence history table 302 of the history information 30 using the wafer ID. Instead of acquiring the crack state information from the history information 30, the information acquisition section 500 may acquire the image information generated by the user's photographing operation or the sketch information generated by the user's drawing operation or the reading operation.

As described above, the crack occurrence process identifying section 501 inputs the crack occurrence state information, acquired by the information acquisition section 500 as the input data, to the learning model 10 in response to the occurrence of the crack in the wafer W, and identifies a process (crack occurrence process) that causes the crack in the wafer W. The crack occurrence process identifying section 501 obtains the scores for the processes S1 to S7 as the output data of the learning model 10, and generates the crack occurrence process information by identifying the crack occurrence process which is the process corresponding to a maximum value of the scores (in the example of FIG. 17, the substrate receiving process S1).

The learned-model storage section 52 is a database that stores the learning model 10 as the learned model to be used in the crack occurrence process identifying section 501. The number of learning models 10 stored in the learned-model storage section 52 is not limited to one. For example, multiple learning models 10 may be stored in the learned-model storage section 52 for different conditions, such as for a machine-learning method, a type of data included in the crack occurrence state information, a type of data included in the crack occurrence process information, etc. These multiple learning models 10 may be selectively used. The learned-model storage section 52 may be a memory section of an external computer (for example, a server type computer or a cloud type computer). In that case, the crack occurrence process identifying section 501 accesses the external computer.

The output processing section 502 performs output processing to output the crack occurrence process information generated by the crack occurrence process identifying section 501. For example, the output processing section 502 may transmit the crack occurrence process information to the user terminal device 6, and the user terminal device 6 may display the display screen based on the crack occurrence process information. The output processing section 502 may transmit the crack occurrence process information to the database device 3, and the crack occurrence process information may be registered in the history information 30.

### (Information Processing Method)

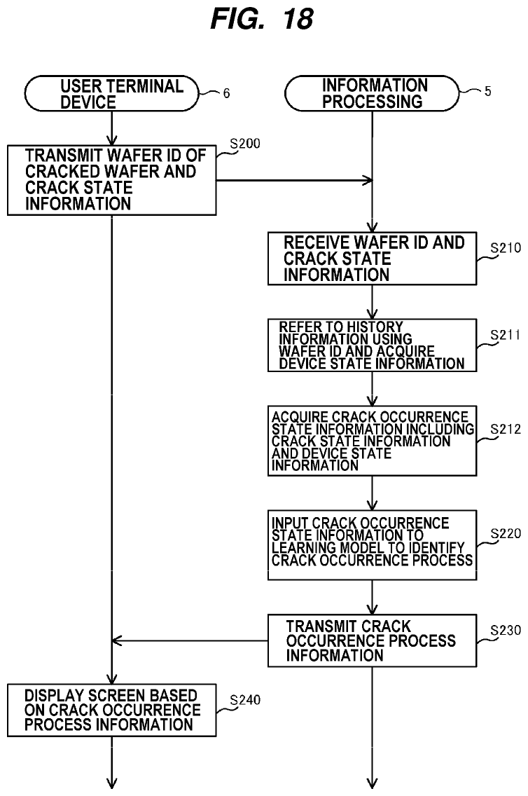

FIG. 18 is a flowchart illustrating an example of an information processing method performed by the information processing device 5. In this embodiment, operations will be described in a case where the user manipulates the user terminal device 6 to analyze the cause of crack in the wafer W when the wafer W is cracked.

First, in step S200, when the user performs an input operation of inputting a wafer ID for identifying a cracked wafer W on the user terminal device 6 and a photographing operation of generating an image of the cracked wafer W with the camera 60, the user terminal device 6 transmits the wafer ID and the image information photographed by the camera 60 to the information processing device 5 as the crack state information.

Next, in step S210, the information acquisition section 500 of the information processing device 5 receives the wafer ID and the crack state information (the image information) transmitted in the step S200. In step S211, the information acquisition section 500 uses the wafer ID received in the step S210 to refer to the process history table 300 and the device-state history table 301 of the history information 30, thereby obtaining the device state information indicating the substrate processing process performed on the cracked wafer W. As a result, in step S212, the information acquisition section 500 obtains the crack occurrence state information including the image information as the crack state information indicating the crack state of the wafer W identified by the wafer ID and the device-state information at the time when the substrate processing process is performed on the wafer W.

Next, in step S220, the crack occurrence process identifying section 501 inputs the crack occurrence state information acquired in the step S210 as input data to the learning model 10, thereby generating, as output data, the crack occurrence process information corresponding to the crack occurrence state information and identifies the crack occurrence process for the wafer W.

Next, in step S230, the output processing section 502 transmits the crack occurrence process information to the user terminal device 6 as an output process for outputting the crack occurrence process information generated in the step S220. The crack occurrence process information may be transmitted to the database device 3 in addition to or instead of the user terminal device 6.

Next, in step S240, upon receiving the crack occurrence process information transmitted in the step S230, the user terminal device 6 displays a display screen based on the crack occurrence process information as a response to the transmission process in the step S200. As a result, the crack occurrence process of the cracked wafer W can be visually recognized by the user. In the above information processing method, the steps S210 to S212 correspond to an information acquisition process, the step S220 corresponds to a crack occurrence process identifying process, and the step S230 corresponds to an output processing process.

As described above, according to the information processing device 5 and the information processing method according to the present embodiment, the crack occurrence state information including the crack state information and the device state information acquired in response to the occurrence of crack in the wafer W is input to the learning model 10, so that the cause of crack in the wafer W can be identified. Therefore, the user can deal with the crack in the wafer W quickly and appropriately without relying on the user's experience or knowledge.

### OTHER EMBODIMENTS

The present invention is not limited to the above-described embodiments, and various modifications can be made and used without deviating from the scope of the present invention. All of them are included in the technical concept of the present invention.

In the above embodiments, the database device 3, the machine-learning device 4, and the information processing device 5 are described as being configured as separate devices, but these three devices may be configured as a single device. In one embodiment, any two of these three devices may be configured as a single device. Further, at least one of the machine-learning device 4 and the information processing device 5 may be incorporated into the control unit 26 of the substrate processing device 2 or the user terminal device 6.

In the embodiments described above, the neural network is employed as the learning model 10 that implements the machine learning performed by the machine-learning section 401, while other machine-learning model may be employed. Examples of the other machine-learning model include tree type (e.g., decision tree, regression tree), ensemble learning (e.g., bagging, boosting), neural network type including deep learning (e.g., recurrent neural network, convolutional neural network, LSTM), clustering type (e.g., hierarchical clustering, non-hierarchical clustering, k-nearest neighbor algorithm, k-means clustering), multivariate analysis (e.g., principal component analysis, factor analysis, logistic regression), and support vector machine.

### (Machine Learning Program and Information Processing Program)

The present invention can be provided in a form of a program (machine learning program) that causes the computer 900 to function as each section of the machine-learning device 4, and in a form of a program (machine learning program) that causes the computer 900 to execute each process of the machine-learning method. Further, the present invention can be provided in a form of a program (information processing program) that causes the computer 900 to function as each section included in the information processing device 5, and in a form of a program (information processing program) that causes the computer 900 to execute each process included in the information processing method according to the above embodiments.

### (Inference Apparatus, Inference Method, and Inference Program)

The present invention can be provided in a form of not only the information processing device 5 (information processing method or information processing program) according to the above embodiments, but also in a form of an inference apparatus (inference method or inference program) used for inferring the crack occurrence process information. In that case, the inference apparatus (inference method or inference program) may include a memory and a processor. The processor may execute a series of processes. The series of processes includes information acquisition processing (information acquisition process) and inference process (inference process). The information acquisition processing (information acquisition process) includes acquiring the crack occurrence state information including the crack state information indicating the crack state when a wafer W is cracked in the substrate processing process performed by the substrate processing device 2 and the device state information indicating the state of the polishing unit 22 when the substrate processing process is performed on the cracked wafer W. The inference process (inference process) includes inferring the process (crack occurrence process) that causes the crack in the wafer W when acquiring the crack occurrence state information in response to the occurrence of the crack in the wafer W. The crack occurrence process inferred is one of the processes S1 to S7 included in the substrate processing process.

The form of the inference apparatus (inference method or inference program) can be applied to various devices more easily than when the information processing device is implemented. It is readily understood by a person skilled in the art that the inference method performed by the crack occurrence process identifying section may be applied with use of the learning model 10 as the learned model generated by the machine-learning device 4 and the machine-learning method according to the above embodiments when the inference apparatus (inference method or inference program) infers the crack occurrence process.

### INDUSTRIAL APPLICABILITY

The present invention is applicable to information processing apparatus, an inference apparatus, a machine-learning apparatus, an information processing method, an inference method, and a machine-learning method.

### REFERENCE SIGNS LIST

1 . . . substrate processing system, 2 . . . substrate processing device, 3 . . . database device, 4 . . . machine-learning device, 5 . . . information processing device, 6 . . . user terminal device, 7 . . . network, 10 . . . learning model, 11 . . . learning data, 20 . . . housing, 21 . . . load-unload unit, 22 . . . polishing unit, 23 . . . substrate transport unit, 24 . . . cleaning unit, 25 . . . film-thickness measuring unit, 26 . . . control unit, 30 . . . history information, 40 . . . control section, 41 . . . communication section, 42 . . . learning-data storage section, 43 . . . learned-model storage section, 50 . . . control section, 51 . . . communication section, 52 . . . learned-model storage section, 60 . . . camera, 201 . . . camera, 220 . . . polishing table, 221 . . . top ring, 222 . . . polishing-liquid supply nozzle, 223 . . . dresser, 224 . . . atomizer, 230A . . . first linear transporter, 230B . . . second linear transporter, 231 . . . swing transporter, 232 . . . lifter, 233 . . . temporary station, 260 . . . control section, 261 . . . communication section, 262 . . . input section, 263 . . . output section, 264 . . . memory section, 300 . . . process history table, 301 . . . device-state history table, 302 . . . crack occurrence history table, 400 . . . learning-data acquisition section, 401 . . . machine-learning section, 500 . . . information acquisition section, 501 . . . crack occurrence process identifying section, 502 . . . output processing section, 900 . . . computer, 2200 . . . polishing pad, 2210 . . . top ring body, 2211 . . . carrier, 2212 . . . membrane, 2212a-2212d . . . membrane pressure chamber, 2213 . . . retainer ring, 2214 . . . retainer-ring airbag, 2214a . . . retainer-ring pressure chamber, 2300 . . . transport hand, 2301 . . . vertical movement mechanism, 2302 . . . horizontal movement mechanism, 2303 . . . retainer-ring station

1. An information processing apparatus comprising:
an information acquisition section configured to acquire crack occurrence state information including crack state information and device state information, the crack state information indicating crack state of a substrate that has been cracked in a substrate processing process performed by a substrate processing device including a polishing unit configured to perform a polishing process on the substrate and a substrate transport unit configured to transport the substrate to and from the polishing unit, the device state information indicating a state of the polishing unit when the substrate processing process is performed on the cracked substrate; and
a crack occurrence process identifying section configured to identify a process that causes the crack in the substrate by inputting the crack occurrence state information acquired by the information acquisition section to a learning model in response to the occurrence of the crack in the substrate, the learning model having been generated by machine learning that causes the learning model to learn a correlation between the crack occurrence state information and crack occurrence process information indicating the process that causes the crack in the substrate, the process being among processes included in the substrate processing process.

2. The information processing apparatus according to claim 1, wherein the crack state information included in the crack occurrence state information includes:
image information indicating the cracked substrate that has been photographed; or
sketch information indicating the cracked substrate that has been sketched.

3. The information processing apparatus according to claim 1, wherein the device state information included in the crack occurrence state information includes the state of the polishing unit which includes at least one of:
a position of a top ring of the polishing unit;
a height of the top ring;
pressure in a pressure chamber provided in the top ring; and
a flow rate of pressurized fluid supplied to the pressure chamber.

4. The information processing apparatus according to claim 1, wherein the device state information included in the crack occurrence state information further includes a state of the substrate transport unit in addition to the state of the polishing unit, the state of the substrate transport unit including at least one of:
a position of the substrate transport unit;
a height of the substrate transport unit; and
presence or absence of the substrate in the substrate transport unit.

5. The information processing apparatus according to claim 1, wherein the crack occurrence process information includes processes included in the substrate processing process, the processes including at least one of:
a substrate receiving process in which the polishing unit receives the substrate from the substrate transport unit before the polishing process;
a pre-polishing oscillation process in which the polishing unit moves the substrate to a polishing position before the polishing process;
a pre-polishing lowering process in which the polishing unit lowers the substrate to a polishing height before the polishing process;
a polishing process in which the polishing unit performs the polishing process on the substrate;
a post-polishing elevating process in which the polishing unit elevates the substrate to a moving height after the polishing process;
a post-polishing oscillation process in which the polishing unit moves the substrate to a transfer position after the polishing process; and
a substrate delivery process in which the polishing unit delivers the substrate to the substrate transport unit after the polishing process.

6. An inference apparatus comprising:
a memory; and
a processor configured to perform:
an information acquisition process of acquiring crack occurrence state information including crack state information and device state information, the crack state information indicating crack state of a substrate that has been cracked in a substrate processing process performed by a substrate processing device including a polishing unit configured to perform a polishing process on the substrate and a substrate transport unit configured to transport the substrate to and from the polishing unit, the device state information indicating a state of the polishing unit when the substrate processing process is performed on the cracked substrate; and
an inferring process of inferring a process that causes the crack in the substrate when acquiring the crack occurrence state information in the information acquisition process in response to occurrence of the crack in the substrate, the process inferred being among processes included in the substrate processing process.

7. A machine-learning apparatus comprising:
a learning-data storage section storing multiple sets of learning data including crack occurrence state information and crack occurrence process information, the crack occurrence state information including crack state information and device state information, the crack state information indicating crack state of a substrate that has been cracked in a substrate processing process performed by a substrate processing device including a polishing unit configured to perform a polishing process on the substrate and a substrate transport unit configured to transport the substrate to and from the polishing unit, the device state information indicating a state of the polishing unit when the substrate processing process is performed on the cracked substrate, the crack occurrence process information indicating a process that causes the crack in the substrate, the process being among processes included in the substrate processing process;
a machine-learning section configured to cause a learning model to learn a correlation between the crack occurrence state information and the crack occurrence process information by inputting the multiple sets of learning data to the learning model; and
a learned-model storage section configured to store the learning model that has learned the correlation by the machine learning section.

8. (canceled)

9. (canceled)

10. (canceled)
